(19) United States
(12) Patent Application Publication
(10) Pub. No.: US 2015/0343139 A1
(43) Pub. Date: Dec. 3, 2015

(54) IMPLANTABLE PUMPS AND RELATED METHODS OF USE

(71) Applicant: Boston Scientific Scimed, Inc., Maple Grove, MN (US)

(72) Inventors: Bryan Allen CLARK, Forest Lake, MN (US); Aiden FLANAGAN, Kilcolgan (IE)

(73) Assignee: Boston Scientific Scimed, Inc.

(21) Appl. No.: 14/716,397

(22) Filed: May 19, 2015

Related U.S. Application Data
(60) Provisional application No. 62/004,959, filed on May 30, 2014.

(51) Int. Cl. A61M 5/142; A61M 5/158; A61M 5/172; A61F 2/82

(52) U.S. Cl. CPC A61M 5/14276; A61F 2/82; A61M 5/158; A61M 5/1723; A61M 2210/1039; A61M 2005/1726; A61M 2230/65; A61M 2005/14284

(57) ABSTRACT

A medical device may include a pump configured to dispense an agent for treating a condition of a lung. The pump may be configured to be implanted subcutaneously into a patient. The medical device may also include a treatment device coupled to the pump and configured to deliver the agent to the lung.

### CROSS-REFERENCE TO RELATED APPLICATION

This patent application claims the benefit of priority to U.S. Provisional Application No. 62/004,959, filed on May 30, 2014, the entirety of which is incorporated herein by reference.

### TECHNICAL FIELD

The present disclosure relates to implantable pump and catheter systems for delivering agents to the lungs and related methods of implantation and use.

### BACKGROUND

Chronic obstructive pulmonary disease (COPD) includes conditions such as, e.g., chronic bronchitis and emphysema. COPD currently affects over 15 million people in the United States alone and is currently the third leading cause of death in the country. The primary cause of COPD is the inhalation of cigarette smoke, responsible for over 90% of COPD cases. The economic and social burden of the disease is substantial and is increasing.

Chronic bronchitis is characterized by chronic cough with sputum production. Due to airway inflammation, mucus hypersecretion, airway hyperresponsiveness, and eventual fibrosis of the airway walls, significant airflow and gas exchange limitations result.

Emphysema is characterized by the destruction of the lung parenchyma. This destruction of the lung parenchyma leads to a loss of elastic recoil and tethering which maintains airway patency. Because bronchioles are not supported by cartilage like the larger airways, they have little intrinsic support and therefore are susceptible to collapse when destruction of tethering occurs, particularly during exhalation.

Acute exacerbations of COPD (AECOPD) often require emergency care and inpatient hospital care. An AECOPD is defined by a sudden worsening of symptoms (e.g., increase in or onset of cough, wheeze, and sputum changes) that typically last for several days, but can persist for weeks. An AECOPD is typically triggered by a bacterial infection, viral infection, or pollutants, which manifest quickly into airway inflammation, mucus hypersecretion, and bronchoconstriction, causing significant airway restriction.

Despite relatively efficacious drugs (long-acting muscarinic antagonists, long-acting beta agonists, corticosteroids, and antibiotics) that treat COPD symptoms, a particular segment of patients known as “frequent exacerbators” often visit the emergency room and hospital with exacerbations and also have a more rapid decline in lung function, poorer quality of life, and a greater mortality risk.

Reversible obstructive pulmonary disease includes asthma and reversible aspects of COPD. Asthma is a disease in which bronchoconstriction, excessive mucus production, and inflammation and swelling of airways occur, causing widespread but variable airflow obstruction thereby making it difficult for the asthma sufferer to breathe. Asthma is further characterized by acute episodes of airway narrowing via contraction of hyper-responsive airway smooth muscle.

The reversible aspects of COPD include excessive mucus production and partial airway occlusion, airway narrowing secondary to smooth muscle contraction, and bronchial wall edema and inflation of the airways. Usually, there is a general increase in bulk (hypertrophy) of the large bronchi and chronic inflammatory changes in the small airways. Excessive amounts of mucus are found in the airways, and semisolid plugs of mucus may occlude some small bronchi. Also, the small airways are narrowed and show inflammatory changes.

In asthma, chronic inflammatory processes in the airway play a central role in increasing the resistance to airflow within the lungs. Many cells and cellular elements are involved in the inflammatory process including, but not limited to, mast cells, eosinophils, T lymphocytes, neutrophils, epithelial cells, and even airway smooth muscle itself. The reactions of these cells result in an associated increase in sensitivity and hyperresponsiveness of the airway smooth muscle cells lining the airways to particular stimuli.

The chronic nature of asthma can also lead to remodeling of the airway wall (i.e., structural changes such as airway wall thickening or chronic edema) that can further affect the function of the airway wall and influence airway hyper-responsiveness. Epithelial denudation exposes the underlying tissue to substances that would not normally otherwise contact the underlying tissue, further reinforcing the cycle of cellular damage and inflammatory response.

In susceptible individuals, asthma symptoms include recurrent episodes of shortness of breath (dyspnea), wheezing, chest tightness, and cough. Currently, asthma is managed by a combination of stimulus avoidance and pharmacology.

Bronchiectasis is a condition where lung airways become enlarged, flabby, and scarred. In the injured areas, mucus often builds up, causing obstruction and/or infections. A cycle of repeated infections may continue to damage the airways and cause greater mucus build-up. Bronchiectasis can lead to health problems such as respiratory failure, atelectasis, and heart failure.

Strategies for managing COPD and other conditions of the lung include smoking cessation, vaccination, rehabilitation, and drug treatments (e.g., inhalers or oral medication). Drug treatments of COPD conditions, such as, e.g., mucus production, inflammation, and bronchoconstriction often suffer from poor patient compliance. That is, certain patients may not accurately administer prescribed doses, reducing the efficacy of treatment. For drug treatments utilizing inhalation, there is also an accompanying drug loss due to upper airway entrapment, which may lead to an over-prescription of active drugs. Also, inhalation treatments can be ineffective at treating smaller airways of the lung (e.g., airways that are smaller than 2 mm). For drug treatments utilizing oral administration, there is an accompanying systemic loss which also leads to an over-prescription of active drugs. The over-prescription of drugs may result in suboptimal treatment and/or a build-up of toxins within the lungs and/or other organ systems. In other situations, drugs may not be deposited evenly to areas of the lungs because of particle size and/or blockage of airways preventing the drugs from reaching distal regions of the lungs (e.g., a heterogeneous delivery of drugs). Blockages may be caused by mucus and narrowing of the airway due to inflammation and remodeling.

Thus, a need exists for drug delivery mechanisms that efficiently and effectively treat diseases of the lungs.

### SUMMARY OF THE DISCLOSURE

The present disclosure relates to implantable pump and catheter systems for delivering agents to the lungs and related methods of implantation and use.

In one aspect, the present disclosure is directed to a medical device. The medical device may include a pump configured to dispense an agent for treating a condition of a lung. The pump may be configured to be implanted within a patient. The medical device may also include a treatment device coupled to the pump and configured to deliver the agent to the lung.

Various examples of the present disclosure may include one or more of the following aspects: wherein the treatment device is configured to be inserted into a first airway of the lung and deliver the agent to airways of the lung that are distal to the first airway; wherein the treatment device further includes an anchor member configured to anchor the treatment device to a lung airway wall; wherein the anchor member is an expandable member configured to appose against the airway wall; wherein the treatment device is an elongate member, and the anchor member is a spiral formed from a length of the elongate member; wherein the anchor member is a stent; wherein the stent includes a plurality of branches extending from a branch point; wherein the anchor member is a hook, barb, spike, or the like, disposed along an outer surface of the treatment device; wherein an outer surface of the treatment device or anchor member includes one or more needles configured to deliver the agent from the pump to the lung airway wall; further including a needle disposed at a distal end of the treatment device, the needle being configured to deliver agent through an airway wall of the lung to a lung parenchyma; wherein the treatment device includes at least one sensor configured to detect indicia of a triggering event, and a controller coupled to the pump and the at least one sensor, the controller being configured to determine the occurrence of the triggering event based upon input from the sensor and dispense agent from the pump after determining that the triggering event has occurred; wherein the controller is further configured to block the dispensation of agent to the lung after the triggering event has ended; wherein the sensor is an impedance sensor; wherein the controller is configured to determine that the triggering event has occurred when the impedance sensor senses an impedance of the lungs that is below an impedance threshold level; and wherein the pump is implanted subcutaneously.

In another aspect, the present disclosure is directed to a method of treating a lung. The method may include implanting a pump within a patient, and directing an agent configured to treat a condition of the lung from the pump toward and/or into the lung.

Various examples of the present disclosure may include the following aspect: wherein the pump is implanted subcutaneously.

In yet another aspect, the present disclosure is directed to a method of treating a lung. The method may include directing an agent to the lymphatic system to treat a condition of the lung.

Various examples of the present disclosure may include one or more of the following aspects: wherein the agent is delivered to a lymph node located distally within the lung; wherein the agent is directed proximally through the lung via the lymphatic system to treat the condition of the lung; wherein the lymph node is one of an intrapulmonic lymph node, peribronchial lymph node, segmental lymph node, or hilar lymph node; and wherein the agent is directed to the lymphatic system via a pump with a container and a sensor, and a treatment device, wherein the pump, the container, the sensor, and the treatment device are implanted within a patient.

### BRIEF DESCRIPTION OF THE FIGURES

The accompanying drawings, which are incorporated in and constitute a part of this specification, illustrate various exemplary embodiments and together with the description, serve to explain the principles of the disclosed embodiments.

FIG. 1 is a schematic view of a lung system and a lymphatic system.

FIG. 2 is a schematic view of a system for treating the lungs according to an embodiment of the present disclosure.

FIGS. 3-12 are side views of treatment devices according to embodiments of the present disclosure that can be used in conjunction with a system, such as the system shown in FIG. 2.

### DETAILED DESCRIPTION

Reference will now be made in detail to embodiments of the present disclosure, examples of which are illustrated in the accompanying drawings. Wherever possible, the same reference numbers will be used throughout the drawings to refer to the same or like parts.

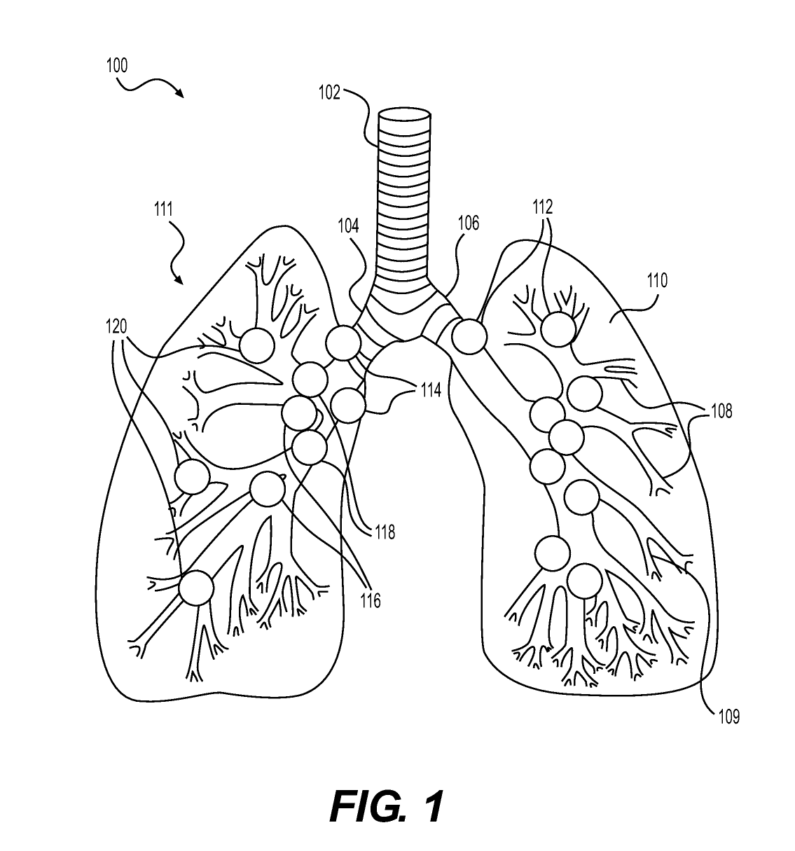

FIG. 1 illustrates a lung system 100 including a trachea 102. As air flows in through the nose and mouth, trachea 102 delivers the air to the lungs for respiratory functions. Trachea 102 divides into two main bronchi—the right primary bronchus 104 and the left primary bronchus 106. In lung system 100, both the right primary bronchus 104 and the left primary bronchus 106 divide into a plurality of bronchi, which further divide into a plurality of smaller airways, including airways 108. Airways 108 may be bronchi, bronchioles, or other suitable airways that may subdivide into smaller and more numerous airways 108 at branch points 109. Branch points 109 may subdivide a given airway 108 into two or more subsequent generation airways 108. Airways 108 may terminate into a plurality of alveoli (not shown) located in lung parenchyma 110. The alveoli are small elastic air sacs which enable gas exchange between the pulmonary system and the circulatory system, including oxygen and CO₂ exchange.

Lung system 100 may be a damaged lung system exhibiting at least one symptom of COPD or another condition of the lung. In some embodiments, one or more airways 108 may be partially or completely blocked due to inflammation and/or excessive mucus production, among other conditions. In some cases, the elastic recoil of alveoli may be lost, and inhaled air may not be completely expelled during exhalation. In some embodiments, lung system 100 may include one or more collateral channels or bypasses (not shown) that facilitate exhalation and gas exchange in the lung.

Lymphatic system 111 may include one or more lymph nodes 112 that may be disposed within or adjacent to lung system 100. Lymph nodes 112 may be oval-shaped organs of the immune system that are linked to one another by lymphatic vessels. Lymphatic system 111 may include intrapulmonic (interlobar) lymph nodes 116, peribronchial lymph nodes 118, and segmental lymph nodes 120 that drain toward hilar lymph nodes 114. Hilar lymph nodes 114 may be located adjacent to both right primary bronchus 104 and left primary bronchus 106, and may drain toward the inferior tracheobronchial (carinal) nodes, superior tracheobronchial nodes, and tracheal nodes (not shown).

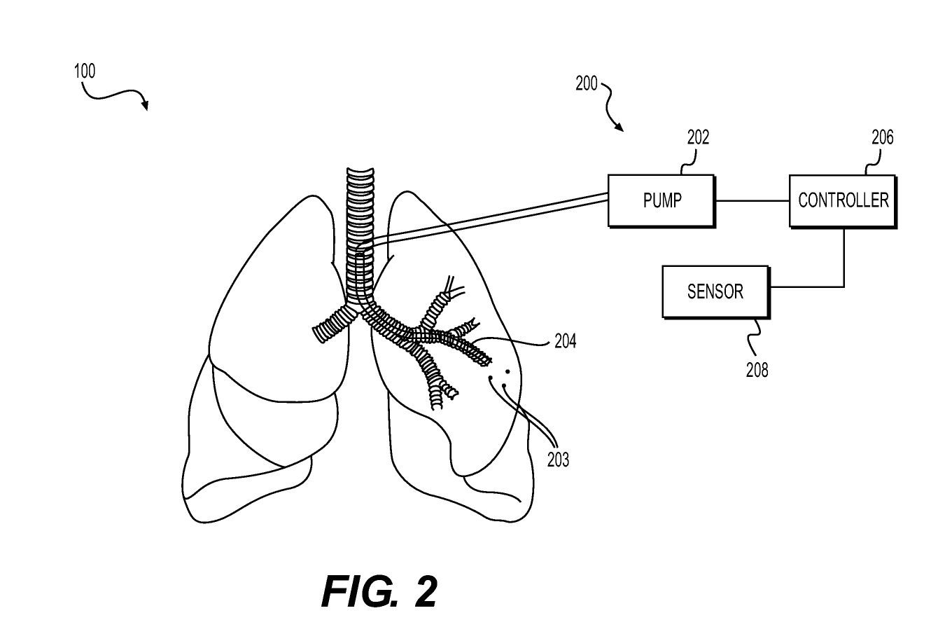

FIG. 2 is an illustration of lung system 100 being treated with a system 200 according to the present disclosure. The system 200 may include a pump 202 with a source of an agent 203, a treatment device 204, a controller 206, and one or more sensors 208. Treatment device 204 may include one or more tools that can be positioned at a treatment site within lung system 100 and/or lymphatic system 111.

Pump 202 may be implanted (e.g., surgically) into a patient by any suitable mechanism. In some embodiments, pump 202 may be implanted beneath a patient's skin (i.e., subcutaneously). Pump 202 may be implanted in the abdomen, lung cavity, peritoneal space, or in another suitable location, and may be secured by any suitable mechanism.

Pump 202 may be an implantable pump, such as, e.g., a peristaltic pump, piston pump, motorized pump, microfluidic pump, infusion pump, or the like. Pump 202 may be powered by electrical power, mechanical power, chemical power, or another suitable mechanism. In one embodiment, pump 202 may be powered by an energy harvesting device that may be energized by, e.g., body movements, breathing, or the like. In some embodiments, pump 202 may include redundant power sources (e.g., multiple batteries). Pump 202 may include a source (e.g., a reservoir) of agent 203 to be delivered to one or more of lung system 100 and lymphatic system 111. In some embodiments, pump 202 may include a plurality of reservoirs to hold a plurality of agents 203. Pump 202 may be refillable via, e.g., a port, valve, septum, or other suitable member that is accessible at or beneath the skin.

Agent 203 may be a drug, pharmaceutical, steroid (e.g., a corticosteroid), or another suitable agent or combination of agents that can be delivered to lung system 100. In some embodiments, agent 203 may be selected from the family of oral or inhaled medications currently available for treatment of COPD, asthma, lung cancer, bronchiectasis, and/or other conditions of the lung. Additionally, agent 203 may be used to treat any other condition of the body. In one exemplary embodiment, agent 203 may be utilized to deliver a flu vaccine over a period of multiple months (e.g., the winter season) to a patient. In some embodiments, agent 203 may include bronchodilators, inhaled steroids, oral steroids, phosphodiesterase-4 inhibitors (e.g., roflumilast), theophylline, antibiotics, or any suitable combination. Bronchodilators, which may otherwise be delivered by inhalation, may include long-acting bronchodilators (tiotropium, salmeterol, formoterol, arformoterol, indacaterol, aclidinium, and the like) or short-acting bronchodilators (albuterol, levalbuterol, ipratropium, and the like). Inhaled steroids may include inhaled corticosteroids that may reduce airway inflammation and help prevent exacerbations (fluticasone, budesonide, and the like). Exemplary combinations of drugs include salmeterol and fluticasone, and formoterol and budesonide. Other agents 203, such as, e.g., carbocisteine, mecysteine, N-acetylcysteine, may be additionally or alternatively utilized. Agent 203 may also include agents directed toward asthma, allergies, cancers, or other ailments of the lung. In some embodiments, agent 203 may include anti-cholinergics, muscarinics, antibiotics, steroids, methylxanthines, and/or alpha anti-trypsin. In some embodiments, agent 203 may be a chemotherapy drug for treating inoperable cancers or tumors of lung system 100 or lymphatic system 111 where metastases frequently occur. In some embodiments, agent 203 may include antibiotics (e.g., erythromycin), antifungals, or antiviral drugs. In some embodiments, antibiotics may be utilized to treat cysts or other infections.

In one embodiment, antibiotics and/or anti-fungals may be delivered to cysts of the lung. In another embodiment, antibiotics and/or mucolytics may be delivered to bronchiectatic regions and/or to patients having Cystic Fibrosis. In another embodiment, anti-rejection drugs may be delivered to patients having one or more transplanted lungs. In another embodiment, anti-inflammatory drugs may be delivered to poorly ventilated areas of the lung, in, e.g., COPD patients.

In some embodiments, agent 203 may be delivered in combination with oxygen to lung parenchyma 110 to improve oxygen supply to the lungs and to reduce air trapping, inflammation, bronchoconstriction, and infection, among other conditions. In such embodiments, an externally communicating port, valve, catheter, or the like may be utilized to attach to an external oxygen supply. However, implantable oxygen supplies are also contemplated. Oxygen delivery may reduce hypoxia and dyspnea.

Pump 202 may be coupled to treatment device 204. In some embodiments, treatment device 204 may extend from pump 202 and extend through trachea 102. However, other suitable entry and exit points to lung system 100 may alternatively be utilized. Treatment device 204 may be a catheter or other suitable member configured to deliver agent 203 from pump 202 to lung system 100 and/or lymphatic system 111. Treatment device 204 may be inserted into a patient by a bronchoscope or other suitable member. Treatment device 204 may include a lubricious coating, or may otherwise be atraumatic so as not to inadvertently puncture tissue during navigation of treatment device 204 within the body.

Agent 203 may be delivered to regions of the lungs exhibiting poor airflow or other conditions of the lung, and regions where conventional inhalers cannot effectively deliver drugs. The regions for delivery of agent 203 can be optimally determined by, e.g., hyperpolarized He MRI scans, among other imaging techniques. Conversely, using similar techniques, agent 203 may be delivered into the regions of the lungs exhibiting healthy airflow in order to deposit agent 203 into the parenchyma, for example, when it is desired to treat healthy lungs, or a tumor.

In some embodiments, treatment device 204 may be advanced through trachea 102 and larger airways 108 to a point where the tip of treatment device 204 is adjacent to a lymph node 112 located distally in the lungs. Then, treatment device 204 may be secured adjacent, or even inserted into, one or more lymph nodes 112. In some embodiments, agent 203 may be delivered to a lymph node 112 that is not located within lung system 100, but distal to lung system 100. Agent 203 administered to a distal lymph node 112, or administered to a lymph node 112 that is distal to the lungs, may move cranially or proximally in the direction of trachea 102, flowing through lung system 100. In some embodiments, as agent 203 is delivered to one or more lymph nodes 112, agent 203 may pass through the lymph system 111 toward trachea 102, and agent 203 may have a therapeutic effect on airways 108 and lung parenchyma 110 in between the region of agent 203 delivery and trachea 102. Delivery of agent 203 via the lymph system may be particularly advantageous in cancer treatments. In some embodiments, agent 203 may be delivered into lung fissures (where the pleural membrane does not penetrate). In some embodiments, agent 203 may be delivered to an upper portion of the lung fissures where agent 203 may flow downward via gravity to fill the fissure space and diffuse into a remainder of the lung. Each lung may have an oblique fissure, and the right lung may have an additional horizontal fissure. In some embodiments, agent 203 may additionally diffuse from the fissure space into the pleural cavity and subsequently to a remainder of the lung.

In some embodiments, treatment device 204 may be inserted into or adjacent to the pleural space (not shown) surrounding lung system 100 to treat a condition of the lung. Agents 203 delivered to the pleural space may diffuse across the visceral pleura into lymphatic system 111, travelling cranially through lymphatic system 111 to provide a therapeutic effect to the lungs as described previously.

In one embodiment, treatment device 204 may be positioned for a localized treatment of bronchiectasis. A mucolytic formulation may be administered by system 200 to break up mucus. Antibiotics may also be administered to combat infections resulting from bronchiectasis. Treatment device 204 also may be positioned for the treatment of pneumonia, localized fungal or bacterial infections, or cysts.

The pump 202 and treatment device 204 may be utilized as a short-term use device, e.g. for several days or weeks post-discharge for patients who have experienced an acute exacerbation of COPD, for localized regions of bronchiectasis, or for other conditions described herein which may resolve with treatment after a short period of time. After the acute condition has resolved, the treatment device 204 may be removed from the patient. In a short time-frame, pump 202 and treatment device 204 may reduce patient time within the hospital, and also may reduce the chance of relapse or subsequent treatment of the same condition. Alternatively, the short-term use pump could be utilized to evaluate whether a patient experiences benefit from the system. If a benefit is identified, a permanent pump 202 and treatment device 204 could be implanted later. The pump for the temporary system could be an external wearable pump similar to pump 202, or could be a different pump system, such as, e.g., a balloon that uses pressure to dispense the agent 203 through the treatment device 204.

In another embodiment, treatment device 204 may treat a larger region of lung system 100 by nebulizing agent 203 entering into the first or second generation bronchi of a lung lobe for general bronchodilation. In yet another embodiment, a large area of the lung may be treated by positioning a distal end of treatment device 204 (e.g., a piercing-type distal end) within a blood vessel (e.g., a branch of a bronchial artery) such that agent 203 may be carried distally to the lung via perfusion and/or blood flow in the blood vessels around the bronchi and bronchioles. In some embodiments, agent 203 may be delivered to particular bronchi, lung regions, or airways 108 where constriction or hyperreactivity, for example, is more pronounced. The particular regions may be identified prior to implantation of treatment device 204 into lung system 100.

In some embodiments, treatment device 204 may be deployed in the upper airways 108 of lung system 100, e.g. in one or both of right primary bronchus 104 and left primary bronchus 106. In some embodiments, multiple treatment devices 204 may be coupled to a single pump 202, while traversing through lung system 100 to different treatment locations. A distal end of treatment device 204 may include an aerisolizer or other suitable drug dispersing tip, so that when agent 203 is administered during an inhalation cycle, agent 203 is able to disperse to distal portions of the lung via the normal flow of air through the lung. In some embodiments, pump 202 and treatment device 204 may be configured to administer agent 203 only during inhalation. In such embodiments, sensor 208 may be used to detect inhalation and exhalation cycles to trigger the administration of agent 203. Sensor 208 may detect respiration cycles, and inhalation starts and stops, via impedance or pressure measurements. The initiation of agent delivery may coincide with the detection of inhalation onset, and agent delivery may end upon the detection of inhalation offset, for example. In some embodiments, the energy, motion, and/or airflow of inhalation may cause the release and/or atomization of agent 203 into the flow of inhaled air. In some embodiments, exhalation may cause the delivery of agent 203 to cease. In one embodiment, a propeller that rotates in one direction only may power this mechanism.

In some embodiments, treatment device 204 may deliver agent 203 via one or more needles to a wall of an upper airway (e.g., trachea, mainstem, or second or subsequent generation bronchi), allowing agent 203 to diffuse into the vascular system or a blood vessel, among other suitable locations. That is, a distal end of treatment device 204 may be beveled or otherwise sufficiently sharp to pierce through tissue. Thus, treatment device 204 may be a needle, cannula, blade, tube, rod, or other suitable member configured to pierce through tissue. Any agent 203 that subsequently diffuses into the arterioles may be transported to the rest of the distal bronchial tree. In some embodiments, treatment device 204 may include a microarray or matrix of needles on an abluminal or outer surface. In some embodiments, treatment device 204 may include one or more needles each being configured to puncture an airway wall and disperse agent 203 through a plurality of perfusing holes disposed along a length of the needle. In some embodiments, agent 203 may be delivered to emphysematic voids or bula. In some embodiments, the voids may be disposed in the upper lobes such that agent 203 delivered to the voids may flow by gravity to other portions of the lung.

In some embodiments, numerous types of drugs may be delivered to the same or to different locations. In one embodiment, a bronchodilator, mucolytic, antibiotic, or another suitable agent 203 may be delivered to a lung airway wall (e.g., via nebulizer or bronchial artery system), while a corticosteroid, anti-inflammatory, anti-protease drug, or another suitable agent 203 may be delivered to the parenchyma 110 (e.g., via the pulmonary artery system). In one embodiment, sensor 208 may detect airway resistance, edema, an inflammatory reaction, or another symptom, causing controller 206 to release one or more agents 203 via treatment device 204 to the lung airways or parenchyma, if desired. In some embodiments, there may be a continuous delivery of agent 203 to the parenchyma, and a selective delivery of agent 203 to the airway. In some embodiments, there may be a continuous delivery of agent 203 to the airway, and a selective delivery of agent 203 to the parenchyma. In some embodiments, there may be continuous or selective delivery of agent 203 to both the airways and parenchyma.

In some embodiments, the controller 206 may include a processor that is generally configured to accept information from the system and system components, and process the information according to various algorithms to produce control signals for controlling pump 202 and treatment device 204. The processor may accept information from the system and system components, process the information according to various algorithms, and produce information signals that may be directed to visual indicators, digital displays, audio tone generators, or other indicators of, e.g., a user interface, in order to inform a user of the system status, component status, procedure status or any other useful information that is being monitored by the system. The processor may be a digital IC processor, analog processor or any other suitable logic or control system that carries out the control algorithms. In some embodiments, controller 206 may record treatment parameters such as, e.g., the volumes of agents 203 administered at different times, sensor data, and other suitable treatment parameters so that they may be accessed for concurrent or subsequent analysis.

In some embodiments, controller 206 may be implanted subcutaneously with pump 202. In some embodiments, controller 206 may be internal to pump 202. Alternatively, controller 206 may be disposed outside of the patient, but otherwise in communication with pump 202 through suitable communication mechanisms such as, e.g., wireless, IR, Bluetooth, or another suitable communication mechanism. In some embodiments, controller 206 may be configured to communicate with other instruments such as, e.g., diagnostic instruments, tablets, computers, cell phones, servers, or other instruments to transmit and receive data, instructions, or other suitable information. The communication of controller 206 with external devices may allow third parties (e.g., care providers or physicians) to observe the health condition of a patient, and additionally to allow third parties to control pump 202 (e.g., increase dosing, decrease dosing, suspend dosing).

A sensor 208 may be in communication with controller 206, and may facilitate delivery of agent 203 from pump 202 and treatment device 204. In some embodiments, sensor 208 may detect when agent 203 should be administered, or if the dosing of agent 203 should be adjusted. For example, pump 202 may be configured to administer an agent 203 (e.g., bronchodilators and/or corticosteroids) at a lower dose for stable-state COPD on a daily, hourly, or continuous basis. However, if one or more sensors 208 detect a triggering event (e.g., increased inflammation, airway resistance, other symptoms of an acute exacerbation, or other triggering events), pump 202 may increase the dose of agent 203 to relieve bronchial smooth muscle contraction, submucosal gland secretions, and/or inflammation, among other symptoms. Controller 206 may continue to dose agent 203 until sensor 208 determines that the symptoms have subsided to an accepted level. Thus, controller 206 may dose agent 203 to the lungs based upon a feedback mechanism, such as, e.g., a PID feedback loop or a fuzzy logic controller, among others.

In one embodiment, sensor 208 may be an impedance sensor. A reduction in impedance over a baseline may indicate that there is a less than normal amount of air in the lungs. The reduction in air may be due to inflamed airways, constricted smooth muscle, and/or mucus in the airways, among other factors. In response to these conditions sensed by sensor 208, controller 206 may direct pump 202 and treatment device 204 to administer a higher dose of agent 203 to relieve these symptoms. In some embodiments, sensor 208 may additionally or alternatively include pH sensors, pressure sensors, temperature sensors, stress/strain gauges, oxygen sensors, carbon dioxide sensors, sensors for other blood/lymph constituents, or other suitable sensors indicating that a triggering event has or may soon occur. The occurrence or imminent occurrence of a triggering event may necessitate the release of one or more agents 203 by system 200. Sensors 208 may be disposed inside or outside of the body. In some embodiments, sensors 208 may be radar sensors.

Alternatively or additionally, all or a portion of system 200 (e.g., one or more of pump 202, treatment device 204, controller 206, and sensors 208) may be formed of a radiopaque material so that it can be visualized under fluoroscopic guidance, or may otherwise include radiopaque or other imaging markers for guidance. The markers may be used to ensure that respective components are properly located within the body. In some embodiments, all or a portion of system 200 may be coated with a substance (e.g., a drug) that helps prevent cell ingrowth that may have an adverse impact on system 200. In some embodiments, all or a portion of system 200 may include a pro-thrombotic to help prevent bleeding. In some embodiments, all or a portion of system 200 may include a material to limit inflammatory reaction and cell growth that could block or otherwise obstruct the delivery of agent 203 to the lungs, and/or an anti-fouling coating.

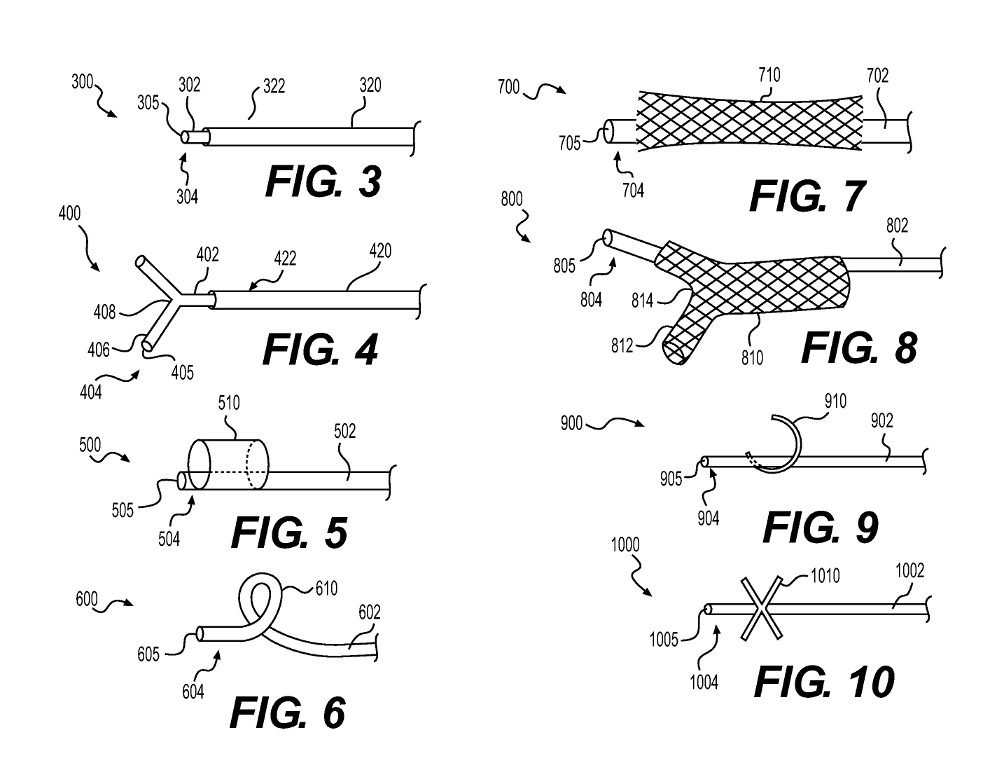

A treatment device 300 is shown in FIG. 3. Treatment device 300 may include an elongate member 302 extending from a proximal end (not shown) toward a distal end 304. An opening 305 may be disposed at distal end 304 of elongate member 302. Opening 305 may be a nozzle, port, or another suitable opening configured to dispense agent 203 into lung system 100 or lymphatic system 111. Agent 203 may be dispensed from opening 305 by aerosol, sprays, jets, trickles, or other suitable mechanisms. That is, agent 203 may be pumped from pump 202 toward opening 305 through one or more lumens of elongate member 302. In some embodiments, multiple lumens may be disposed through elongate member 302 in communication with opening 305 to allow for the selective delivery of different agents 203 through one or more openings 305. In some embodiments, the multiple lumens may connect pump 202 to additional openings 305. In some embodiments, multiple openings 305 may be disposed at distal end 304, and/or along elongate member 302. In some embodiments, treatment device 300 may be a needle or may otherwise be sufficiently sharp to pierce through tissue as described with reference to treatment device 204. Additionally or alternatively, one or more needles may be disposed along the surface of elongate member 302, each of the one or more needles being in communication with pump 202 via the lumen of elongate member 302. Elongate member 302 may be formed of any suitable biocompatible material, including polymers, metals, alloys, or the like.

In some embodiments, one or more inflatable/expandable members (e.g., balloons) may be disposed along elongate member 302 to limit the direction and/or regions of the delivery of agent 203. For example, in one embodiment, one or more inflatable members may be utilized to expand and seal against an airway wall proximal to opening 305, to limit the delivery of agent 203 to portions of a lung airway that are distal to opening 305. In another embodiment, one or more inflatable members at different axial positions distal and proximal to opening 305 may be utilized to limit the delivery of agent 203 to an isolated region of a lung airway.

Elongate member 302 may extend distally from and be longitudinally translatable with respect to a second elongate member 320 that extends from a proximal end (not shown) toward a distal end 322. Elongate member 302 may be disposed through a lumen of second elongate member 320. Second elongate member 320 may be any suitable bronchoscopic member, such as, e.g., a bronchoscope, that is configured to deliver elongate member to a desired location within lung system 100 (referring to FIGS. 1 and 2). Elongate member 320 may be removed after the delivery and implantation of elongate member 302.

A treatment device 400 is shown in FIG. 4. Treatment device 400 may include an elongate member 402 extending from a proximal end (not shown) toward a distal end 404. Treatment device 400 may include a plurality of openings 405 that are each disposed at one of a plurality of branches 406. Branches 406 may be disposed at distal end 404 of elongate member 402, and each branch 406 may extend distally from a branch point 408 disposed along elongate member 402. As shown in FIG. 4, elongate member 402 is depicted with two branches 406, but may include any additional number of branches. Elongate member may be formed from substantially the same materials as elongate member 302, and opening 405 may be substantially similar to opening 305 described with reference to FIG. 3. In some embodiments, additional lumens may be disposed through one or more of branches 406. Each additional lumen may be in communication with a respective opening 405 to allow for the selective delivery of different agents 203. In some embodiments, multiple openings 405 may be disposed along or at the distal end of each branch 406. In some embodiments, different branches 406 may be placed in different airways. In one embodiments, one branch 406 may be placed in an airway, while a separate branch 406 is delivered through an airway wall (e.g., punctures the airway wall).

Elongate member 402 may extend distally from and be longitudinally translatable with respect to a second elongate member 420 that extends from a proximal end (not shown) toward a distal end 422. Elongate member 420 may be substantially similar to elongate member 320 described with reference to FIG. 3.

A treatment device 500 is shown in FIG. 5. Treatment device 500 may include an elongate member 502 extending from a proximal end (not shown) toward a distal end 504. An opening 505 may be disposed at distal end 504 of elongate member 502. Elongate member 502 may be substantially similar to elongate member 302, but may include one or more anchor members 510. Opening 505 may be substantially similar to opening 305 described with reference to FIG. 3.

Anchor member 510 may be disposed at distal end 504, and/or at one or more suitable locations along elongate member 502, and may be configured to anchor treatment device 500 to a target treatment location within the lung airways or parenchyma. In the embodiment shown in FIG. 5, anchor member 510 may be a cylindrical member having a larger diameter than elongate member 502. Anchor member 510 may be biased toward an expanded configuration shown in FIG. 5 such that anchor member 510 does not permit elongate member 502 to move while anchor member 510 is in the expanded configuration. That is, in the expanded configuration, anchor member 510 may exert a radially outward force to the walls of the lung airways. This apposition of anchor member 510 to the walls of the lung airways may prevent the movement of treatment device 500 without, e.g., surgical intervention. In some embodiments, the outer surface of anchor member 510 of may include spikes, protrusions, barbs, hooks, or have other suitable features, such as, e.g., tacky coatings, to facilitate the anchoring of anchor member 510 to lung airways. In some embodiments, one or more needles may be disposed along the outer surface of anchor member 510 to deliver agent 203 to the surface of a lung airway wall.

A treatment device 600 is shown in FIG. 6. Treatment device 600 may include an elongate member 602 extending from a proximal end (not shown) toward a distal end 604. An opening 605 may be disposed at distal end 604 of elongate member 602. Elongate member 602 may be substantially similar to elongate member 302, but may include one or more anchor members 610. Opening 605 may be substantially similar to opening 305 described with reference to FIG. 3.

Anchor member 610 may be disposed at distal end 604, and/or at one or more suitable locations along elongate member 602, and may be configured to anchor treatment device 600 to a target treatment location within the lung airways or parenchyma. In the embodiment shown in FIG. 6, anchor member 610 may be a spiral or coil formed from a length of elongate member 602. Once deployed, anchor member 610 may appose against a lung airway wall, preventing the movement of treatment device 600 without, e.g., surgical intervention. Anchor member 610 may have similar features for anchoring and/or delivery of agent 203 as described with reference to anchor member 510. Anchor member 610 may be substantially straight when constrained during delivery in a delivery device, such as, e.g., elongate member 320 described with reference to FIG. 3, but may expand upon placement within an airway to provide a force against the airway wall. Any additional number of anchor members 610 may be disposed along elongate member 602. Any suitable material may be utilized to construct anchor member 610, such as, e.g., polymers, metals, shape-memory materials, and mechanical fixation materials. In one embodiment, anchor member 610 may be formed of Nitinol.

A treatment device 700 is shown in FIG. 7. Treatment device 700 may include an elongate member 702 extending from a proximal end (not shown) toward a distal end 704. An opening 705 may be disposed at distal end 704 of elongate member 702. Elongate member 702 may be substantially similar to elongate member 302, but may include one or more anchor members 710 that are attached to elongate member 702. Opening 705 may be substantially similar to opening 305 described with reference to FIG. 3.

Anchor member 710 may be disposed at distal end 704, and/or at one or more suitable locations along elongate member 702, and may be configured to anchor treatment device 700 to a target treatment location within the lung airways or parenchyma. In the embodiment shown in FIG. 7, anchor member 710 may be a stent, basket, mesh, globe, nest, or other suitable member disposed around a length of elongate member 702. Anchor member 710 may be biased toward the expanded configuration shown in FIG. 7 such that anchor member 710 does not permit elongate member 702 to move while anchor member 710 is in the expanded configuration. That is, in the expanded configuration, anchor member 710 may exert a radially outward force to the walls of the lung airways. This apposition of anchor member 710 to the walls of the lung airways may prevent the movement of treatment device 700 without, e.g., surgical intervention. Anchor member 710 may have similar features for anchoring and/or delivery of agent 203 as described with reference to anchor member 510. For example, a plurality of needles may be disposed along the surface of stent 710 in order to deliver agent 203 to the lungs via pump 202 and one or more lumens of elongate member 702.

A treatment device 800 is shown in FIG. 8. Treatment device 800 may include an elongate member 802 extending from a proximal end (not shown) toward a distal end 804. An opening 805 may be disposed at distal end 804 of elongate member 802. Elongate member 802 may be substantially similar to elongate member 302, but may include one or more anchor members 810 that are attached to elongate member 802. Opening 805 may be substantially similar to opening 305 described with reference to FIG. 3.

Anchor member 810 may be disposed at distal end 804, and/or at one or more suitable locations along elongate member 802, and may be configured to anchor treatment device 800 to a target treatment location within the lung airways or parenchyma. In the embodiment shown in FIG. 8, anchor member 810 may be a stent disposed around a length of elongate member 802. Anchor member 810 may be substantially similar to anchor member 710 described with reference to FIG. 7, except that anchor member 810 may include a plurality of branches 812 extending from a branch point 814 that is disposed along anchor member 810. That is, anchor member 810 may be Y-shaped such that branch point 814 can appose against a bodily branch point (such as a branch point 109 described with reference to FIG. 1). As shown in FIG. 8, anchor member 810 is depicted with two branches 812, but may include any additional number of branches. In the embodiment shown in FIG. 8, treatment device 800 includes one elongate member 802. However, any suitable number of elongate members 802 may be utilized, or an elongate member with a corresponding shape to anchor member 810 (e.g., elongate member 402) may alternatively be utilized.

A treatment device 900 is shown in FIG. 9. Treatment device 900 may include an elongate member 902 extending from a proximal end (not shown) toward a distal end 904. An opening 905 may be disposed at distal end 904 of elongate member 902. Elongate member 902 may be substantially similar to elongate member 302, but may include one or more anchor members 910. Opening 905 may be substantially similar to opening 305 described with reference to FIG. 3.

Anchor member 910 may be disposed at distal end 904, and/or at one or more suitable locations along elongate member 902, and may be configured to anchor treatment device 900 to a target treatment location within the lung airways or parenchyma. In the embodiment shown in FIG. 9, anchor member 910 may be a partially-circular, staple-like, and/or hook-shaped member. Once deployed, anchor member 910 may pierce through a lung airway wall, preventing the movement of treatment device 900 without, e.g., surgical intervention. Anchor member 910 may have similar features for anchoring and/or delivery of agent 203 as described with reference to anchor member 510. In some embodiments, the ends of anchor member 910 may be open such that anchor member 910 also serves as a needle for delivering agent 203 through a lung airway wall, while simultaneously anchoring treatment device 900. Additional anchor members 910 may be located along elongate member 902. In some embodiments, anchor member 910 may be substantially straight during delivery in a delivery device, e.g., elongate member 320 described with reference to FIG. 3, but may expand to the curved or hook-shaped configuration shown in FIG. 9 upon deployment in an airway. To remove elongate member 902 from the airway after a period of implantation, the anchor member 910 may be collapsed when an outer sheath (not shown) is extended over the elongate member 902 and distally past the anchor member 910. The outer sheath may cause the disengagement of anchor member 910 from the airway tissue, and allow the elongated member 902 to be removed from the airway.

A treatment device 1000 is shown in FIG. 10. Treatment device 1000 may include an elongate member 1002 extending from a proximal end (not shown) toward a distal end 1004. An opening 1005 may be disposed at distal end 1004 of elongate member 1002. Elongate member 1002 may be substantially similar to elongate member 302, but may include one or more anchor members 1010. Opening 1005 may be substantially similar to opening 305 described with reference to FIG. 3.

Anchor member 1010 may be disposed at distal end 1004, and/or at one or more suitable locations along elongate member 1002, and may be configured to anchor treatment device 1000 to a target treatment location within the lung airways or parenchyma. In the embodiment shown in FIG. 10, anchor member 1010 may include a plurality of barbs. Once deployed, anchor member 1010 may pierce through a lung airway wall, preventing the movement of treatment device 1000 without, e.g., surgical intervention. Anchor member 1010 may have similar features for anchoring and/or delivery of agent 203 as described with reference to anchor member 510. In some embodiments, the ends of anchor member 1010 may be open such that anchor member 1010 also serves as a needle for delivering agent 203 through a lung airway wall, while simultaneously anchoring treatment device 1000. Additional anchor members 1010 may be located along elongate member 1002. In some embodiments, anchor member 1010 may be substantially straight during delivery in a delivery device, e.g., elongate member 320 described with reference to FIG. 3, but may expand to the configuration shown in FIG. 10 upon deployment in an airway. An outer sheath (not shown) similar to the outer sheath described with reference to FIG. 9 may be utilized to disengage anchor member 1010 from the airway tissue.

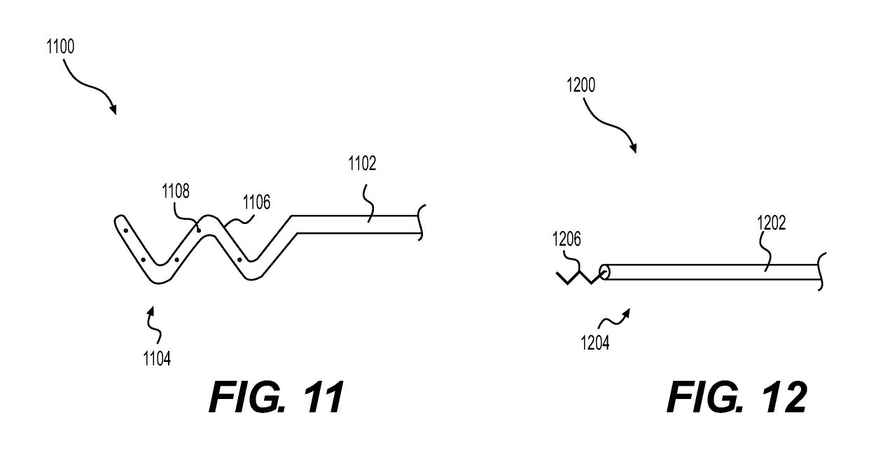

A treatment device 1100 is shown in FIG. 11. Treatment device 1100 may include an elongate member 1102 extending from a proximal end (not shown) toward a distal end 1104. Elongate member 1102 may be substantially similar to elongate member 302, but may include an anchor member 1106 formed at distal end 1104. Anchor member 1106 may be coil-shaped, spiral-shaped, sinusoidal-shaped, wavy-shaped, or otherwise suitable shaped to appose itself against lung airway walls. One or more openings 1108 may be disposed along the side surfaces of anchor member 1106 to dispense agent 203 to the lungs. In some embodiments, an opening 1108 may be disposed at distal end 1104 in addition or alternative to the openings 1108 on the side surfaces of anchor member 1106.

Once deployed, anchor member 1106 may support itself against lung airway walls, preventing the movement of treatment device 1100 without, e.g., surgical intervention. Anchor member 1106 may have similar features for anchoring and/or delivery of agent 203 as described with reference to anchor member 510. In some embodiments, anchor member 1106 may be substantially straight during delivery in a delivery device, e.g., elongate member 320 described with reference to FIG. 3, but may expand to the configuration shown in FIG. 11 upon deployment in an airway.

A treatment device 1200 is shown in FIG. 12. Treatment device 1200 may include an elongate member 1202 extending from a proximal end (not shown) toward a distal end 1204. Elongate member 1202 may be substantially similar to elongate member 302, but may include an anchor member 1206 extending from distal end 1204. Anchor member 1206 may be formed as a coil (e.g., a screw) that may pierce through lung airway walls. Anchor member 1206 may be hollow or formed as a solid. One or more openings (not shown) may be disposed along the side surfaces of anchor member 1206 to dispense agent 203 to the lungs. In some embodiments, an opening may be disposed at a distal end of anchor member 1206 in addition or alternative to the openings on the side surfaces of anchor member 1206. Anchor member 1206 may be formed of Nitinol, stainless steel, or another suitable material. In some embodiments, anchor member 1206 may have a shape memory. In some embodiments, anchor member 1206 also may have a sharp tip for penetration into the lung airway walls.

Once deployed, anchor member 1206 may pierce through lung airway walls, preventing the movement of treatment device 1200 without, e.g., surgical intervention. Anchor member 1206 may have similar features for anchoring and/or delivery of agent 203 as described with reference to anchor member 510. In some embodiments, anchor member 1206 may be substantially straight when constrained in elongate member 1202, but may expand to the configuration shown in FIG. 12 upon deployment in an airway.

A flexible strain relief region proximal to the anchor members of the above-referenced anchor members may be utilized to reduce the probability of breathing movement dislodging a given anchor member. In some embodiments, the flexible strain relief region may be formed of a softer material that is able to stretch. In some embodiments, the flexible strain relief region may be formed by one or more coils or non-straight features.

Any aspect set forth in any embodiment may be used with any other embodiment set forth herein. The devices and apparatus set forth herein may be used in any suitable medical procedure, and may be advanced through any suitable body lumen and body cavity. For example, the apparatuses and methods described herein may be used through any natural body lumen or tract, or through incisions in any suitable tissue. In particular, any feature described with reference to treatment devices 204 and 300-1200 are interchangeable with one another.

It will be apparent to those skilled in the art that various modifications and variations can be made in the disclosed systems and processes without departing from the scope of the disclosure. Other embodiments of the disclosure will be apparent to those skilled in the art from consideration of the specification and practice of the disclosure disclosed herein. It is intended that the specification and examples be considered as exemplary only. The following disclosure identifies some other exemplary embodiments.

We claim:

1. A medical device, comprising:
a pump configured to dispense an agent for treating a condition of a lung, wherein the pump is configured to be implanted within a patient; and
a treatment device coupled to the pump and configured to deliver the agent to the lung.

2. The medical device of claim 1, wherein the treatment device is configured to be inserted into a first airway of the lung and deliver the agent to airways of the lung that are distal to the first airway.

3. The medical device of claim 1, wherein the treatment device further includes an anchor member configured to anchor the treatment device to a lung airway wall.

4. The medical device of claim 3, wherein the anchor member is an expandable member configured to appose against the airway wall.

5. The medical device of claim 3, wherein the treatment device is an elongate member, and the anchor member is a spiral formed from a length of the elongate member.

6. The medical device of claim 3, wherein the anchor member is a stent.

7. The medical device of claim 3, wherein the stent includes a plurality of branches extending from a branch point.

8. The medical device of claim 3, wherein the anchor member is a hook, barb, or spike disposed along an outer surface of the treatment device.

9. The medical device of claim 3, wherein an outer surface of the treatment device or anchor member includes one or more needles configured to deliver the agent from the pump to the lung airway wall.

10. The medical device of claim 1, further including a needle disposed at a distal end of the treatment device, the needle being configured to deliver agent through an airway wall of the lung to a lung parenchyma.

11. The medical device of claim 1, further including at least one sensor configured to detect indicia of a triggering event, and a controller coupled to the pump and the at least one sensor, the controller being configured to determine the occurrence of the triggering event based upon input from the sensor and dispense agent from the pump after determining that the triggering event has occurred.

12. The medical device of claim 11, wherein the controller is further configured to block the dispensation of agent to the lung after the triggering event has ended.

13. The medical device of claim 11, wherein the sensor is an impedance sensor.

14. The medical device of claim 13, wherein the controller is configured to determine that the triggering event has occurred when the impedance sensor senses an impedance of the lungs that is below an impedance threshold level.

15. A method of treating a lung, the method comprising:
implanting a pump within a patient; and
directing an agent configured to treat a condition of the lung from the pump toward and/or into the lung.

16. A method of treating a lung, the method comprising:
directing an agent to the lymphatic system to treat a condition of the lung.

17. The method of claim 16, wherein the agent is delivered to a lymph node located distally within the lung.

18. The method of claim 17, wherein the agent is directed proximally through the lung via the lymphatic system to treat the condition of the lung.

19. The method of claim 16, wherein the lymph node is one of an intrapulmonic lymph node, peribronchial lymph node, segmental lymph node, or hilar lymph node.

20. The method of claim 16, wherein the agent is directed to the lymphatic system via a pump with a container and a sensor, and a treatment device, wherein the pump, the container, the sensor, and the treatment device are implanted within a patient.
